(19) United States
(12) Patent Application Publication
(10) Pub. No.: US 2011/0086074 A1
(43) Pub. Date: Apr. 14, 2011

(54) COMBINATIONS OF NIACIN AND AN OXICAM

(75) Inventors: Pradeep Jairao Karatgi, Hyderabad (IN); Thilek Kumar Muniyappan, Ranipet (IN); Shantanu Yeshwant Damle, Thane (West) (IN); Sesha Sai Marella, Guntur (IN); Harshal Prabhakar Bhagwatwar, Hyderabad (IN); Raviraj Sukumar Pillai, Hyderabad (IN); Ish Kumar Khanna, Alpharetta, GA (US)

(73) Assignees: DR. REDDY'S LABORATORIES LTD., Hyderabad, 500 016, Andhra Pradesh (IN); DR. REDDY'S LABORATORIES, INC., Bridgewater, NJ (US)

(21) Appl. No.: 12/995,711

(22) PCT Filed: Jun. 2, 2009
(86) PCT No.: PCT/US09/45923

§ 371 Date: Dec. 2, 2010

Related U.S. Application Data
(60) Provisional application No. 61/094,161, filed on Sep. 4, 2008.

(30) Foreign Application Priority Data
Jun. 2, 2008 (IN) 1345/CHE/2008

(51) Int. Cl. A61K 31/549; A61K 9/00; A61P 29/00; A61P 3/06; A61P 3/10

(52) U.S. Cl. 424/400; 514/226.5

(57) ABSTRACT

Pharmaceutical formulations comprising a combination of niacin and an oxicam NSAID, for oral administration, and methods of preparing the formulations.

An aspect of the present invention relates to combinations of niacin and an oxicam drug. In embodiments, the invention includes pharmaceutical formulations comprising a combination of niacin and meloxicam for oral administration. Also included are processes for preparing such formulations and methods of using such formulations in reducing niacin-induced insulin resistance and in the treatment of conditions associated with diabetes and dyslipidemia. Methods of using the formulations of the invention in reducing niacin-induced flushing are also included.

Type 2 diabetes is a metabolic disorder that is primarily characterized by insulin resistance, relative insulin deficiency and hyperglycemia. Insulin resistance is defined as a decreased response of peripheral tissues to insulin action. Increasingly, insulin resistance has been recognized as an integral feature of a metabolic syndrome, which includes glucose intolerance, insulin resistance obesity, dyslipidemia, hypertriglyceridemia, low levels of high density lipoprotein (“HDL”) cholesterol, hypertension and accelerated atherosclerosis. Hyperinsulinemia and delayed clearance of glucose in an oral glucose tolerance test are the hallmarks of insulin resistance in patients.

Niacin (also called nicotinic acid or 3-pyridinecarboxylic acid) is a white, crystalline powder, very soluble in water, with structural Formula I.

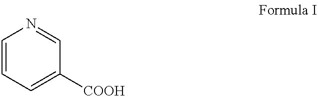

Niacin, when taken in large doses, blocks or inhibits lipolysis in adipose tissue thus reducing free fatty acids in plasma. Niacin is used in the treatment of dyslipidemia because it reduces the synthesis of triglycerides (TG) and enhances secretion of very low density lipoprotein (VLDL), and low density lipoprotein (LDL). Niacin inhibits uptake of apolipoprotein A1 (apoA1) by the liver without affecting the clearance of cholesterol associated with HDL. Niacin has been shown to reduce levels of total cholesterol (TC), LDL and TG. It has also been shown to increase HDL levels in circulation and reduce cardiovascular risk in patients with documented cardiovascular disease.

Commercially, niacin is available in immediate release (IR) formulations as well as sustained release (SR) formulations and intermediate release formulations. Niacin IR is generally more effective than other niacin products for increasing HDL-C (high density lipoprotein-cholesterol). The extent of the lipid reduction varies due to differing baseline levels. Niacin IR therapy should be initiated slowly, with a maximum daily dose of 3 grams. The following description highlights various IR and SR products available commercially along with their doses and efficacy in the treatment of dyslipidemia, the information being based on the report of the Niacin Product Selection Workgroup titled “Veterans Administration Niacin Product Selection,” United States Department of Veterans Affairs, Veteran Health Administration, Aug. 31, 1999 (obtained at the web site www.pbm.va.gov).

Niacin (using a nonprescription IR product, Rugby Laboratories) at a dose of 2-3 grams/day decreased LDL-C by 16-22% and TG by 39-42%, and increased HDL-C by 31-35%.

Niacin (using a prescription IR product NICOLAR™, Rhone-Poulenc Rorer) at a dose up to 3 grams/day decreased LDL-C by 28% and TG by 38%, and increased HDL-C by 22%. In an additional study, NICOLAR at a dose up to 3 grams/day decreased LDL-C by 25% and TG by 26%, and increased HDL-C by 36%. In a third study, NICOLAR at an average dose of 2.25 grams/day decreased LDL-C by 16% and TG by 29%, and increased HDL-C by 27%.

Niacin (using a nonprescription IR product, Goldline Laboratories) at a dose of 3 grams/day decreased LDL-C by 2% and TG by 29%, and increased HDL-C by 25%.

Niacin (using a Kos Pharmaceuticals IR product manufactured for research purposes only) at a dose of 1.5-3 grams/day decreased LDL-C by 13-21% and TG by 19-24%, and increased HDL-C by 10-24%.

Commercial niacin SR products are available as nonprescription products as well as by prescription.

Niacin SR is generally more effective at decreasing LDL-C than niacin IR, although less effective at increasing HDL-C. Niacin SR should be initiated at approximately one-half of the niacin IR dose; the maximum daily dose is 2 grams.

Niacin (using a nonprescription SR product, Goldline Laboratories) at a dose of 1.5-2 grams/day reduced LDL-C by 22%-33% and TG by 25-30%, and increased HDL-C by 13%-17%.

Niacin (using a nonprescription SR product NICOBIDTM, Armour Pharmaceuticals) at a dose of 3 grams/day reduced LDLC by 17% and TG by 2%, and increased HDL-C by 8%. In another study, NICOBID at a dose of 1-2 grams niacin/day reduced LDL-C by 16% and TG by 11%, and increased HDL-C by 12%.

Niacin (using a nonprescription SR product SLO-NIACIN™, Upsher-Smith) at a dose of grams/day reduced LDL-C by 18% and TG by 29%, and increased HDL-C by 16%. In a retrospective study, SLO-NIACIN at an average daily dose of 1.5 grams of niacin, reduced LDL-C by 24% and TG by 33%, and increased HDL-C by 6%.

Niacin (using a nonprescription SR product ENDUR-ACIN™, Endurance Products Corporation) at a dose of 1.5 grams/day reduced LDL by 16%, and increased TG by 4% and HDL-C by <1%. In another study, ENDUR-ACIN at a dose of 1.5-2 grams/day reduced LDL-C by 15-22% and TG by 10-25%, and increased HDL-C by 9-16%. The same study examined differences between younger (20-49 years old) and older (50-70 year old) patients. At a dose of 1.5-2 grams/day, LDL-C was reduced by 29% and TG by 21%, and HDL-C was increased by 8% in older patients; in younger patients LDL-C was reduced by 16%, TG increased by <1% and HDLC increased by 7%. In a third study, ENDUR-ACIN at a dose of 1.5-2 grams/day reduced LDL-C by 20-26% and TG by 9-11%, and increased HDL-C by 4-9%.

Niacin (using a nonprescription SR product, Rugby Inc.) at a dose of 1.2 grams/day reduced LDL-C 6%, and increased TG 11% and HDL-C 2%.

Niacin in the form of a prescription intermediate release product NIASPAN™, Kos Pharmaceuticals, has equivalent daily dosing and similar efficacy to niacin IR. Patients should initiate therapy with the dosing starter pack (Niaspan 375 mg, 500 mg, and 750 mg tablets, each at bedtime for one week and increase ≦500 mg in a four week period).

NIASPAN, at a dose of 1.5 grams niacin at bedtime reduced LDL-C by 13% and TG by 10%, and increased HDL-C by 19%. In a second study, Niaspan up to 3 grams niacin at bedtime reduced LDL-C by 18% and TG by 26%, and increased HDL-C by 32%. A third study examined Niaspan at a dose of 1-2 grams niacin/day at bedtime, reducing LDL-C by 6-15% and TG by 21-28%, and increasing HDL-C by 17-23%.

NIASPAN is indicated as an adjunct to diet for the reduction of elevated TC, LDL-C, apolipoprotein B and TG levels, and to increase HDL in patients with primary hypercholesterolemia and mixed dyslipidemia. NIASPAN® is also indicated to reduce the risk of recurrent nonfatal myocardial infarction and to slow the progression or promote the regression of atherosclerotic disease. NIASPAN is to be taken at bedtime, after a low-fat snack, and doses are individualized according to patient response.

By lowering VLDL levels, niacin also increases the level of HDL in the blood and therefore it is often prescribed for the patients with low HDL, who are also at a high risk of heart attack.

High doses of niacin have been shown to elevate fasting blood sugar levels, thereby worsening type 2 diabetes. Accordingly, niacin is contra-indicated for persons with type 2 diabetes. The mechanism behind niacin-induced insulin resistance and diabetes is presently unknown. However, it is believed that insulin actions are mediated by insulin receptors present in tissues that utilize glucose and it is conceivable that impaired receptor signaling in the presence of niacin may contribute to niacin-induced insulin resistance.

Unfortunately, patients taking pharmacological doses of niacin (ranging from 0.5-3 grams per day) often experience side effects that can include one or more of dermatological complaints (facial flushing and itching, dry skin, and skin rashes including acanthosis nigricans). Facial flushing is the most commonly reported side effect of niacin and is so severe that a significant number of patients discontinue niacin treatment as a result of this severe flushing. The flushing has been shown to be caused by the result of cutaneous vasodilation resulting from niacin-induced release of prostaglandins (e.g., PGD2) in the skin.

What is needed, therefore, is a way to provide the cardiovascular benefits of niacin (lowering of TG and LDL-C and increased HDL-C) while reducing the niacin-mediated increased insulin resistance and facial flushing. Some methods have been proposed to address the facial flushing, but do not adequately address the niacin-induced insulin resistance.

U.S. Pat. Nos. 5,126,145, 6,080,428, 6,129,930, 6,406,715, 6,469,035, 6,676,967, 6,746,691, 6,818,229, and 7,011,848 disclose sustained release formulations of nicotinic acid.

U.S. Pat. Nos. 5,773,453 and 5,981,555, U.S. Patent Application Publication Nos. 2004/0053975 and 2005/0148556, and International Application Publication Nos. WO 2004/103370, WO 2006/017354, WO 2007/041499 and WO 2004/111047 describe methods of reducing niacin-induced flushing.

U.S. Pat. No. 6,406,715 describes an intermediate release nicotinic acid formulation. Conventional niacin therapy has notable limitations that include flushing, most often seen with IR niacin formulations and hepatotoxicity associated with SR niacin formulations, along with reduced flushing when compared with the IR formulation. These adverse effects are related to the absorption rate of niacin from different products when administered orally and its subsequent metabolism (J. A. Pieper, “Understanding niacin formulations,” American Journal of Managed Care, 8:S308-S314, 2002). Niacin is metabolized by two pathways, Phase I (the oxidation route) is via enzyme catalyzed metabolism leading to hepatotoxic metabolites (referred to as “Phase I metabolism”), and Phase II (the conjugation route) leading to metabolites like nicotinuric acid (“NUA”) that cause flushing. In the case of IR niacin formulations, all of the niacin saturates the Phase I metabolism allowing drug to be available for metabolism via the Phase II route leading to severe flushing. On the other hand, SR formulations release the active slowly, causing the niacin to be subjected preferentially to Phase I metabolism, preferably over a period of time, and thus a reduced amount of niacin is available for Phase II metabolism. SR niacin formulations thus causes severe dose-limiting hepatotoxicity by providing a continuous supply of niacin for Phase I metabolism, generating significant quantities of hepatotoxic metabolites while concomitantly resulting in a reduced incidence of flushing. Thus, IR niacin formulations are associated with “high flushing” and “low hepatotoxicity” and the conventional SR niacin formulations are associated with “low flushing” and “high hepatotoxicity”. NIASPAN strikes a balance with the intermediate release formulation by providing a product with low niacin-induced flushing and low hepatotoxicity when administered as a single 1 to 3 g dose orally.

Further, U.S. Pat. No. 6,406,715 discloses that the majority of nicotinic acid in the formulation is released into the blood stream of a human being in about 5 to about 9 hours. Thus, the liver is not exposed to constant levels of nicotinic acid, which results during the administration of long-term, spaced daily doses of SR nicotinic acid. Such intermediate release nicotinic acid formulations are unlikely to cause individuals to develop dose-limiting hepatotoxicity when used as a single daily dose administered in a therapeutic amount. FIG. 3 in the patent depicts a plot of percent drug absorbed (calculated by the Wagner-Nelson method) versus time, wherein it is shown that about 100% of the nicotinic acid is absorbed in vivo within about 7.3 hours after NIASPAN is ingested orally. To achieve this kind of in vivo absorption profile, NIASPAN should release more than about 90% of the contained niacin within about 5 to about 7 hours after oral ingestion.

Most of the documents and reports available in the literature indicate a pressing need for attempts to minimize flushing caused by niacin therapy. The availability of NIASPAN provides a product with reduced hepatotoxicity but does not completely address the flushing induced by niacin. Additionally, there is a need to overcome the insulin-resistance associated with niacin therapy. In placebo-controlled clinical trials for NIASPAN, flushing episodes, i.e., warmth, redness, itching and/or tingling, were the most common treatment emergent adverse events reported by as many as 88% of patients. It is also recommended that NIASPAN be administered at bedtime probably to allow the occurrence of flushing during sleep. Patients are also advised that flushing of the skin may be reduced in frequency or severity by pretreatment with aspirin (taken 30 minutes prior to NIASPAN dose) or non-steroidal anti-inflammatory drugs (e.g., ibuprofen). Flushing varies in severity and may last for several hours after dosing.

There remains a need for a formulation that provides niacin with significantly reduced flushing while also showing low hepatotoxicity and helping overcome the insulin resistance associated with niacin therapy.

Since flushing is observed over several hours post-niacin dosing, the ideal anti-flushing agent should have a suitable terminal elimination half-life in the plasma to be available for modulating flushing over the duration of niacin release. NSAID drugs from the “oxicam” class of compounds have now been surprisingly found to have the desired characteristics. A few commercially available compounds from this class include meloxicam, piroxicam, and tenoxicam. Other oxicam drugs include droxicam and lornoxicam. For simplification, the discussion in this specification will focus on meloxicam, but it is not intended to limit the scope to that particular drug.

Meloxicam, an oxicam derivative, is a non-steroidal anti-inflammatory drug (NSAID). Meloxicam (structural Formula II) has a chemical name 4-hydroxy-2-methyl-N-(5-methyl-2-thiazolyl)-2H-1,2-benzothiazine-3-carboxamide-1,1-dioxide.

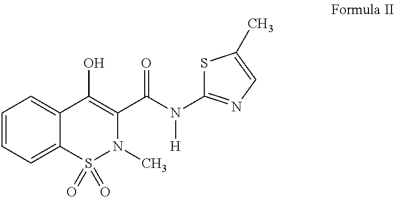

Meloxicam is a pastel yellow solid, practically insoluble in water, with higher solubility observed in strong acids and bases. It is very slightly soluble in methanol. Meloxicam has an apparent partition coefficient of 0.1 in n-octanol/pH 7.4 buffer, and has pKa values of 1.1 and 4.2.

Meloxicam is the active ingredient in products sold as MOBIC® by Boehringer Ingelheim Pharmaceuticals, Inc. in the form of tablets for oral administration containing 7.5 mg or 15 mg of meloxicam, and as an oral suspension containing 7.5 mg of meloxicam per 5 mL. MOBIC products are prescribed for relief of the signs and symptoms of osteoarthritis and rheumatoid arthritis, as well as pauciarticular or polyarticular course juvenile rheumatoid arthritis in patients 2 years of age and older. For the treatment of osteoarthritis and rheumatoid arthritis, the recommended adult oral dose of meloxicam is 7.5 mg or 15 mg, once daily. For the treatment of juvenile rheumatoid arthritis, the recommended oral dose of meloxicam is 0.125 mg/kg of body weight once daily, up to a maximum dose of 7.5 mg for children weighing at least 60 kg.

Meloxicam has been shown to significantly decrease symptoms of pain and stiffness in patients, with a low incidence of gastrointestinal side effects. In models, it exhibits anti-inflammatory, analgesic and antipyretic activities. Its mechanism of action may be related to COX inhibition. Meloxicam has been known to inhibit COX-2 preferentially over COX-1. Thus, meloxicam may demonstrate lower side effects as compared to other NSAIDs, including lowered risk of gastrointestinal bleeding.

### SUMMARY

Embodiments of the present invention relate to combinations of niacin and an oxicam NSAID. Specific embodiments of the invention relate to pharmaceutical formulations comprising a combination of niacin and meloxicam and at least one pharmaceutically acceptable carrier, for oral administration. Also included are processes for preparing such formulations and the methods of using such formulations in reducing insulin resistance and in the treatment of conditions associated with diabetes and dyslipidemia. Methods of using the formulations of the invention in reducing niacin-induced flushing are also included.

In an aspect of the invention, combinations comprise therapeutically effective amounts of niacin and therapeutic to sub-therapeutic anti-inflammatory adult doses of oxicam NSAIDs.

An embodiment of the present invention provides combinations comprising therapeutically effective doses of niacin and therapeutic to sub-therapeutic anti-inflammatory adult doses of meloxicam. In particular embodiments, meloxicam is provided at sub-therapeutic adult anti-inflammatory doses.

In an embodiment, the combination contemplates pharmaceutical formulations comprising therapeutically effective amounts of niacin and meloxicam for oral administration along with a pharmaceutically acceptable carrier.

In another embodiment, the combination formulations comprise unit-dose pharmaceutical formulations having niacin and meloxicam.

In a further embodiment, the combination formulations comprise kits comprising separate formulations of niacin and meloxicam to allow easier administration.

Another aspect of the present invention provides pharmaceutical formulations comprising low-dose meloxicam and a therapeutically effective amount of niacin for oral administration.

In a further embodiment, the combinations of the present invention comprise an IR formulation of niacin and an IR formulation of therapeutically effective anti-inflammatory doses of meloxicam.

In a further embodiment, combination formulations of the present invention comprise a modified release (MR) formulation of niacin and an IR formulation of therapeutically effective anti-inflammatory doses of meloxicam.

In another embodiment combination formulations of the present invention comprise a modified release formulation of niacin and low doses of meloxicam ranging from 0.5 to 15 mg.

In certain aspects, the modified release formulations release the contained niacin at a slower rate into an aqueous fluid than an IR formulation, but at a faster rate than extended release and sustained release formulations known in the art, when tested under similar conditions. In other aspects, the modified release formulations of niacin release niacin at rates similar to NIASPAN or other sustained release formulations known in the art.

In another embodiment, pharmaceutical formulations comprise therapeutically effective anti-inflammatory doses of meloxicam for reducing flushing provoked by the oral administration of niacin to a subject in need thereof.

In another embodiment, pharmaceutical formulations comprise a low dose of meloxicam for reducing the flushing provoked by the oral administration of niacin to a subject in need thereof.

The present invention, in a further aspect, relates to sequential release pharmaceutical formulations comprising meloxicam and modified release niacin, wherein a significant amount of the meloxicam is released prior to the release of a therapeutically significant amount of niacin.

The present invention, in a further aspect, relates to sequential release pharmaceutical formulations comprising meloxicam and immediate release niacin, wherein significant amounts of the meloxicam are released prior to the release of a therapeutically significant amount of niacin

The present invention, in a further aspect, relates to simultaneous release pharmaceutical formulations comprising immediate release meloxicam and modified release niacin, wherein meloxicam and niacin release may commence simultaneously, but niacin release is extended over as long as 8 hours

In further aspects of the invention, substantially all of the meloxicam is released before more than about 50% of the niacin is released from the formulations.

Further provided are methods of reducing flushing provoked by oral niacin administration to a subject in need thereof, wherein the method comprises orally administering to the subject a combination comprising therapeutically effective amounts of niacin and meloxicam.

In an embodiment, a method of reducing flushing provoked by oral niacin administration to a subject in need thereof comprises orally administering to the subject a combination formulation comprising a low-dose of meloxicam and a therapeutically effective amount of niacin.

In an embodiment, a method of reducing flushing provoked by oral niacin administration to a subject in need thereof comprises orally administering to the subject a sequential release pharmaceutical formulation comprising meloxicam and modified release niacin, wherein substantially all of the meloxicam is released prior to the release of a substantial amount of niacin into an aqueous fluid.

In an embodiment, a method of reducing flushing provoked by oral niacin administration to a subject in need thereof comprises orally administering to the subject a sequential release pharmaceutical formulation comprising meloxicam and immediate release niacin, wherein substantially all of the meloxicam is released prior to the release of a substantial amount of niacin into an aqueous fluid.

In certain embodiments, the pharmaceutical formulations of the present invention contain “low-doses” of meloxicam, as compared to the anti-inflammatory adult doses that are typically administered, such low doses being, for example, about 0.5 mg to about 5 mg, about 1 mg to about 5 mg, or about 1 mg to about 4 mg of meloxicam, and also contain about 250 mg to about 3 grams, or about 250 mg to about 1 gram, about 500 mg to about 1 gram, or about 500 mg to about 750 mg, of niacin, to be administered daily.

In other embodiments, the pharmaceutical formulations of the present invention contain therapeutically effective anti-inflammatory doses of meloxicam, for example, about 7.5 mg to about 15 mg, together with about 250 mg to about 2 grams, or about 500 mg to about 1 gram of niacin, to be administered daily.

In other embodiments, the pharmaceutical formulations of the present invention contain therapeutically effective anti-flushing doses of meloxicam, for example, about 3 mg to about 6 mg, together with about 1 gram of niacin, to be administered daily.

In certain embodiments of the invention, weight ratios of meloxicam to niacin in the pharmaceutical formulations range between about 1:100 to about 1:750, about 1:150 to about 1:600, about 1:200 to about 1:500, or about 1:250 to about 1:400.

Methods of reducing the insulin resistance associated with niacin therapy comprising administration of the combinations of the invention are also encompassed by the invention. Processes for the preparation of the formulations are also included.

### DETAILED DESCRIPTION

The present invention relates to combinations of niacin and meloxicam. In embodiments, the invention relates to pharmaceutical formulations comprising a combination of niacin and meloxicam for oral administration. The invention also relates to processes for preparing such formulations and methods of using such formulations in reducing insulin resistance and in the treatment of conditions associated with diabetes and dyslipidemia. Methods of using the formulations of the invention in reducing niacin-induced flushing are also included.

The pharmaceutical formulations of the present invention address an unmet medical need by providing combination formulations comprising therapeutically effective amounts of niacin and therapeutic to sub-therapeutic levels of oxicam-NSAIDs. Such oxicam-NSAIDs include drug substances such as for example meloxicam, piroxicam, and tenoxicam. All other compounds from the oxicam class are also within the scope of this invention.

An embodiment of the present invention provides combinations comprising therapeutically effective amounts of niacin and therapeutic to sub-therapeutic amounts of meloxicam. In an aspect of this embodiment, meloxicam is provided at sub-therapeutic anti-inflammatory doses.

Using low doses of meloxicam will alleviate the dose-dependent adverse effects of meloxicam as well as reduce niacin-induced flushing when administered orally to subjects in need thereof. Further, the immediate and modified release niacin formulations can reduce niacin-induced hepatotoxicity. This will additionally lead to enhanced patient compliance resulting from a better tolerance and reduction in side effects.

It is expected that meloxicam, a preferential COX 2 inhibitor, when combined with niacin therapy, should effectively prevent or minimize the occurrence and/or severity of niacin-induced flushing.

The term “niacin” includes niacin free acid, its prodrugs, and/or its pharmaceutically acceptable salts, solvates, hydrates, enantiomers, polymorphs, and mixtures thereof. As used herein, the term “prodrug” encompasses compounds other than niacin itself which the body metabolizes into niacin, thus producing the same effects as described herein. For example, the compounds include, but are not limited to, nicotinamide, nicotinyl alcohol tartrate, d-glucitol hexanicotinate, aluminum nicotinate, niceritrol, and d,1-alpha-tocopheryl nicotinate.

The term “meloxicam” includes meloxicam and its pharmaceutically acceptable salts, solvates, hydrates, enantiomers, polymorphs, and mixtures thereof.

“Combination,” or “pharmaceutical formulation,” or formulation,” in the context of the present invention refers to any of:

a) A unit dose pharmaceutical formulation comprising niacin and meloxicam.

b) Separate formulations of niacin and meloxicam packaged together in the form of a kit for ready administration.

c) Separate formulations of niacin and meloxicam to be administered together or sequentially.

A combination of niacin and meloxicam as described herein provides one or more of the following advantageous characteristics:

a) Significant reduction in niacin-induced flushing when used with meloxicam, at low-doses as well as at therapeutically effective anti-inflammatory doses of meloxicam.

b) Prevention or reduction in the insulin resistance caused by niacin therapy.

c) An improved gastro-intestinal safety profile, as compared to the currently marketed doses of meloxicam.

d) A reduced niacin-induced hepatotoxicity when compared with the sustained release formulations known in the art or comparable with that of NIASPAN.

e) Improved dyslipidemia or antiatherosclerotic profile as compared to niacin monotherapy

A “unit dose” pharmaceutical formulation as described herein comprises any of:

a) A core comprising immediate release or modified release niacin and optionally a barrier coating applied onto the niacin core, an enteric coating covering the niacin core, optionally a barrier coating applied on the enteric coating and an immediate release formulation of meloxicam coated on the enteric/barrier coating. An outer film coating may optionally be applied to cover the meloxicam layer.

b) A multiparticulate system comprising immediate release meloxicam particles or granules or blend along with pharmaceutically acceptable excipients or tablets and immediate release or modified release niacin particles or granules or blends along with pharmaceutically acceptable excipients or tablets, optionally coated with an enteric polymer which can further be filled into capsules or compressed into tablets using suitable pharmaceutical additives. Optional barrier coatings may also be present as desired.

c) A capsule comprising an immediate release or modified release formulation of niacin which is optionally coated with a seal coating agent (alternatively described as a barrier coating in certain embodiments), said capsule being optionally coated with an enteric coating agent followed by a coating of meloxicam onto the enteric coating. There also may be an optional barrier coating over the enteric coating before the meloxicam is layered on top. Optionally an outer film coating may be applied to cover the meloxicam layer.

d) Bilayered, tri-layered or multi-layered tablets, with at least one layer comprising meloxicam and at least one layer comprising niacin wherein the niacin is either immediate release or modified release, and optionally an intermediate layer which forms a barrier between two active layers.

e) A monolithic tablet wherein immediate release or modified release niacin powders or granules or particles are in intimate mixture with meloxicam powders or granules along with other pharmaceutically acceptable excipients. Optional barrier or enteric layers may be present as desired.

A “kit,” in the context of the present invention, includes two or more formulations arranged in a package, which may be further provided with specific instructions to the patients about the mode of administration of these formulations, e.g., substantially simultaneous ingestion, with no significant time gap between the ingestions of niacin and oxicam NSAID,. Particularly, a meloxicam formulation may be administered together with a niacin formulation. The formulations present in such kits comprise immediate and/or modified release formulations such as tablets, capsules, granules/particles/pellets, and the like including combinations thereof.

In certain embodiments, the present invention envisages a “combination of niacin and meloxicam” per se, without regard to the type of formulations (immediate release or modified release), the form of formulations (a unit dose formulation or a kit comprising two or more separate unit dose formulations for niacin or meloxicam), the sequence of administration, and the doses of each of niacin and meloxicam as long as they are safe for administration to a subject in need thereof.

As used herein, “subject” refers to an animal, such as a mammal, that may benefit from the administration of the compositions or formulations of the present invention. Most often, the subject is a human.

The terms “active” or “active agent” or “active substance” or “active ingredient” or “drug” are used synonymously with niacin or meloxicam or their salts, solvates, hydrates, enantiomers, polymorphs or mixtures thereof, in the description of the invention.

The term “modified release” may be construed synonymously as “slow release” or “extended release” or “delayed release” or “controlled release” or “programmed release” or “pulsed release,” as is known to a skilled person. Such modified release pharmaceutical formulations of the present invention exhibit a slow rate of drug release as compared to an IR formulation (i,e., a formulation having an in vitro release of more than about 75% of the contained active in less than about 2 hours, or in about 1 hour, when tested using a customary dissolution testing method), typically releasing the active beyond about 2 hours up to about 24 hours.

As used herein, a “therapeutically effective amount” is an amount that has a reasonable risk to benefit ratio for the treatment of certain diseases in the subjects in need thereof. In some embodiments, an “anti-flushing effective amount of meloxicam” in the pharmaceutical formulations of the present invention is about 0.1 mg, about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, or an effective anti-inflammatory dose. An “effective anti-inflammatory dose” of meloxicam in the present invention is an amount corresponding to its approved adult anti-inflammatory doses of 7.5 mg or 15 mg, daily. In certain embodiments, the pharmaceutical formulations of the present invention contain “low-doses” of meloxicam, for example, about 0.5 mg to about 6 mg, or about 1 mg to about 5 mg, to be administered daily. A “therapeutically effective amount of niacin,” in the context of the present invention, includes about 250 mg, about 500 mg, about 750 mg, about 1 gram, or about 2 grams of niacin daily.

An aspect of the present invention provides therapeutically beneficial combinations comprising a therapeutically effective amounts of niacin and an anti-flushing effective amount of meloxicam.

In an embodiment, the combination contemplates pharmaceutical formulations comprising a therapeutically effective amount of niacin and an anti-flushing effective amount of meloxicam, for oral administration.

In another embodiment, the combination formulation comprises unit-dose pharmaceutical formulations comprising niacin and meloxicam.

In a further embodiment, combination formulations comprise two separate unit dose formulations, one being a modified release niacin composition of the present invention and the other containing immediate release meloxicam, in the form of a kit for simultaneous administration.

A combination formulation comprises, in some embodiments, meloxicam and modified release niacin formulations. In an embodiment, a modified release formulation releases the contained niacin into an aqueous fluid at a slower rate than its IR formulation, when tested under similar conditions.

In some embodiments, the modified-release niacin formulations of the present invention release the contained niacin in an extended release manner or a delayed release manner, or a combination of delayed and extended release manners.

In the context of the present invention, the desired in vitro and/or in vivo drug release of niacin contained in the pharmaceutical formulation can be achieved by various means, such as, but not limited to, use of surfactants/solubilizers, complexing agents, altering the hardness of the granules/tablets, use of suitable binders in required concentrations, use of hydrophobic and/or hydrophilic pharmaceutical excipients, altering the particle sizes and/or polymorphic form of the active, use of pharmaceutically acceptable coating excipients, and the like, or combinations thereof.

It has been observed that significantly low doses of oxicams like meloxicam (as compared to the normally indicated meloxicam anti-inflammatory doses of 7.5 mg and 15 mg once daily) are highly effective in preventing or minimizing niacin-induced flushing when given orally at a particular dose and a specific manner as described herein. Meloxicam can be provided as a pretreatment, or alternatively can be co-administered with modified or immediate release niacin formulations of the present invention. Further, meloxicam can be provided as separate unit dosage forms before administration of modified release niacin formulations of the present invention, or can be compounded with niacin formulations, wherein an effective amount of meloxicam is released prior to release of niacin so as to block niacin induced flushing.

Another aspect of the present invention provides pharmaceutical formulations comprising low-dose meloxicam and a therapeutically effective amount of niacin for oral administration.

In another embodiment, pharmaceutical formulations comprise a low dose of meloxicam for reducing flushing provoked by the oral administration of niacin to a subject in need thereof.

The present invention, in a further aspect, relates to sequential release pharmaceutical formulations comprising a meloxicam component and a modified or immediate release niacin component, wherein an effective amount of meloxicam component is released prior to release of significant amount of niacin component so as to help block niacin-induced flushing. The term “sequential release” as used herein refers to an in vitro and/or in vivo drug release pattern exhibited by the combination formulations of the present invention, wherein under certain conditions, an effective amount of contained meloxicam (e.g., more than about 40%, about 50%, about 70% or about 90%) is released prior to the release of an significant amount (e.g., less than about 30%, about 20%, or about 10%) of contained niacin, to block the flushing.

Also included are in vitro or in vivo drug release patterns exhibited by the combination formulations of the present invention, wherein meloxicam is released prior to the release of niacin. In certain embodiments, the concept of sequential release also contemplates the commencement of release of niacin prior to the release of meloxicam.

In an embodiment, the niacin is released over a prolonged time, beginning after a delay of about 10 minutes, about 20 minutes, about 30 minutes, about 1 hour, about 2 hours, about 4 hours, or about 8 hours following meloxicam release from the formulation. These parameters can be determined using an in vitro test that simulates oral administration, in which a dosage form is immersed into an acidic environment having pH values below about 4 for about 1 hour, or about 2 hours, for meloxicam release, followed by immersing into a higher pH environment such as at least about 5, or al least about 6, for niacin release.

In vitro testing is only an approximate simulation of the drug dissolution that will occur in vivo, after oral administration. The actual pH environments that are encountered by a formulation begin with the acidic region of the stomach, and transit of a formulation into a higher pH region (due to gastric emptying) occurs at different times after administration, depending on factors such as the presence of food, etc. It is generally desired that some fraction of the meloxicam will dissolve from a formulation, before dissolution of niacin commences. Systemic absorption of both meloxicam and niacin is rapid, following dissolution of the drug from a formulation.

In embodiments of the present invention, when the combination of drugs is orally administered to a human, a substantial part of the contained meloxicam (e.g., more than about 60%, 70%, 80%, or 90%) will be released and absorbed into plasma before about 50% of the contained niacin is released and absorbed.

In another aspect, the contained meloxicam is substantially completely released before about 50% of the contained niacin is released from the formulation.

In a yet further aspect, substantially all of the meloxicam is released from the formulation before less than 25% of niacin is released from the formulation.

In embodiments, the meloxicam will be in immediate release form, not being pH-dependent, and the niacin will be released in higher pH environments, such as pH values greater than about 5, or about 6.

The sequence of administration of two or more separate formulations in a kit as described herein is in some embodiments non-specific. In embodiments, they are substantially simultaneously administered to a subject in need thereof.

The terms “effective amount” or “substantially” as used herein when referring to the release of meloxicam are intended to refer to the release and absorption of amounts of meloxicam sufficient to reduce or block the flushing associated with the absorption of niacin. Thus, any release and absorption profile of meloxicam which results in a reduced or inhibited flushing associated with the administration and subsequent absorption of niacin are within the scope of this application without limitation.

The pharmaceutical formulations of the present invention comprising niacin and meloxicam can be provided as two or more separate unit dosage formulations, each containing niacin and meloxicam separately, or alternatively as part of a combination formulation wherein both of these exhibit desired in vitro and in vivo release profiles.

Alternatively, as described above, niacin and meloxicam may form a part of a combination formulation. Such combination formulations can be provided by various means such as bi-layered, tri-layered, or multi-layered tablets, formulations comprising a plurality of particles (synonymously, beads, granules, or pellets), multiple mini-tablets or mini-capsules formulations, modified release niacin tablets or capsules or particles coated with a meloxicam layer, and the like. These and other formulations described herein as combinations are described in detail herein.

In some specific embodiments, pharmaceutical formulations of the present invention provide meloxicam in an immediate release form and niacin in a modified release form, such that the formulations, when administered to a subject in need thereof, provide therapeutic plasma levels of meloxicam before significant build of niacin in plasma, so as to prevent or minimize niacin-induced flushing. One of the means to achieve this target includes meloxicam pretreatment prior to niacin therapy, that is, sequentially administering to a subject an immediate release meloxicam formulation and, after a specific time delay of about 5 minutes, about 15 minutes, about 30 minutes, about 1 hour, about 1.5 hour, about 2 hours, about 3 hours, about 4 hours, etc., further administering the same subject a modified release formulation of niacin as contemplated herein. The meloxicam formulations for such pretreatment can be prepared by techniques known to a person skilled in the art.

Another means for providing niacin and meloxicam in the desired manner include administering to a subject a modified release formulation of niacin that exhibits a delay of about 30 minutes, about 1 hour, about 1.5 hour, about 2 hours, about 3 hours, or about 4 hours before the release of niacin. Such delay in the release of niacin from its modified release formulation can be accomplished by different techniques such as reservoirs formed with acid-resistant enteric coatings or barrier coatings, matrix formulations that exhibit initial lag in drug release, and the like or combinations thereof. All such niacin formulations are generally included within the purview of modified release formulations of the present invention. The modified release niacin formulations may further be combined with the meloxicam component or may be provided separately in the form of a kit as described above. Whether ingested separately or concurrently in the form of a combination formulation, these pharmaceutical formulations comprising niacin and meloxicam would provide the desired sequential release of meloxicam followed by niacin, and the corresponding in vivo absorption profiles of the two compounds to result in the synergistic effect.

In some embodiments of the present invention, weight ratios of meloxicam to niacin in the pharmaceutical formulations range between about 1:100 to about 1:750, about 1:150 to about 1:600, about 1:200 to about 1:500, or about 1:250 to about 1:400.

The unit combination doses may vary, containing from about 400 mg to about 1 g of niacin and about 2mg to about 10 mg of meloxicam. Maximum combination dosing per day may include about 0.5 g-3.5 g of niacin and about 1-15 mg of meloxicam. Examples of combination doses per day include about 1-3 g of niacin and about 4-12 mg of meloxicam. Further examples of combination doses per day include about 1.5-3 g of niacin and less than about 8 mg of meloxicam. The dosing may be 1-3 times per day, or once daily.

The present invention also relates to pharmaceutical formulations comprising modified and immediate release niacin formulations, wherein the formulations exhibit reduced hepatotoxicity as compared to prior sustained release formulations, or substantially equivalent to that of the NIASPAN product, when tested in vivo under similar conditions.

Further provided are methods of reducing flushing provoked by oral niacin administration to a subject in need thereof, wherein the methods comprise orally administering to the subject a combination formulation comprising therapeutically effective amounts of niacin and meloxicam for oral administration. Such a combination formulation can be provided by one of many different formulation approaches including, for example, any of:

1) Cores comprising immediate release or modified release niacin and optionally a barrier coating applied onto the niacin core, an enteric coating covering the niacin core, optionally a barrier coating applied onto the enteric coating and an immediate release formulation of meloxicam coated onto the enteric/barrier coating. An outer film coating may optionally be applied to cover the meloxicam layer.

2) Multiparticulate systems comprising immediate release meloxicam particles or granules or blends together with pharmaceutically acceptable excipients or tablets and immediate release or modified release niacin particles or granules or blends together with pharmaceutically acceptable excipients or tablets, optionally coated with an enteric polymer, filled into capsules or compressed into tablets using suitable pharmaceutical additives. Optional barrier coatings may also be present as desired.

3) Capsules comprising an immediate release or modified release formulation of niacin which is optionally coated with a seal coating agent (also alternatively described as a barrier coating in certain embodiments), said capsule being optionally coated with an enteric coating agent followed by a coating of meloxicam onto the enteric coating. There also may be an optional barrier coating over the enteric coating before the meloxicam is layered on top. Optionally an outer film coating may be applied to cover the meloxicam layer.

4) Bilayer, tri-layer or multi-layer tablets, with at least one layer comprising meloxicam and at least one layer comprising niacin, wherein the niacin is in immediate release or modified release form, and optionally an intermediate layer forming a barrier between two active layers.

5) Monolithic tablets wherein immediate release or modified release niacin powders or granules are in intimate mixture with meloxicam powders or granules, together with other pharmaceutically acceptable excipients. Optional barrier or enteric layers may be present as desired.

It is to be understood that all the different ways in which delayed and controlled release formulations of niacin can be prepared are within the scope of this application. So also any means by which a combination formulation of niacin and meloxicam can be prepared are within the scope of this application as long as the combination formulation and the release of niacin provide some or all of the therapeutic benefits described herein.

In an embodiment, a method of reducing flushing provoked by oral niacin administration to a subject in need thereof comprises orally administering to the subject a combination formulation comprising low-dose meloxicam and a therapeutically effective amount of niacin.

In a further embodiment, a method of reducing flushing provoked by oral niacin administration to a subject in need thereof comprises orally administering to the subject a sequential release pharmaceutical formulation comprising a meloxicam component and modified release niacin component, wherein the meloxicam component is released preferentially over release of the niacin component into gastrointestinal fluid.

In embodiments, the use of meloxicam particles having mean particle sizes of about 1 μm to about 200 μm, about 3 μm to about 100 μm, or about 5 μm to about 50 μm, are contemplated. Similarly, niacin particles having mean particle sizes of about 10 μm to about 1000 μm, or about 50 μm to about 700 μm, for the preparation of pharmaceutical formulations are contemplated. Such particles of the actives exhibit required micromeritic properties such as but not limited to bulk density, tapped density, angle of repose, Carr index, compressibility ratio, and the like.

In an embodiment, a mean average particle size of meloxicam is less than about 10 μm, for preparation of a formulation.

As used herein, the term “mean particle size” refers to a distribution of particles wherein about 50 volume percent of all particles measured have particle sizes less than the defined mean particle size value, and about 50 volume percent of all measurable particles measured have particle sizes greater than the defined mean particle size value; this can be denoted by the term “D₅₀.” Similarly, a particle size distribution where 90 volume percent of the particles have sizes less than a specified size is referred to as “D₉₀” and a distribution where 10 volume percent of particles have sizes less than a specified size is referred to as “D₁₀.” The desired particle size range material is obtained directly from a synthesis process or any known particle size reduction processes can be used, such as but not limited to sifting, milling, micronization, fluid energy milling, ball milling, and the like. Methods for determining D₁₀, D₅₀ and D₉₀ include laser diffraction, such as using Malvern Instruments Ltd. (Malvern, Worcestershire, United Kingdom) laser diffraction equipment.

Therapeutically effective amounts of actives can be provided in the form of pharmaceutical formulations as a single dose, in multiple doses, or as a partial dose in the form of tablets, capsules, granules (synonymously, “beads” or “particles” or “pellets”), suspensions, emulsions, powders, dry syrups, and the like. All such formulations are included herein without limitation.

Granules can be formed by any processes, using operations such as one or more of dry granulation, wet granulation, extrusion-spheronization, and the like. In an embodiment, the granulation of the active(s), optionally with one or more pharmaceutically acceptable excipients like diluents or fillers, is carried out in equipment such as planetary mixers, rapid mixer granulators (RMG), fluid bed processors and the like. A fluid bed processor with a top spray attachment has been found to be particularly useful. In general, granulation can be carried out by dissolving or dispersing the active ingredient in an organic solvent, optionally with a binder and/or solubilizer, and spraying the solution onto a substrate comprising pharmaceutically acceptable excipients. The granules obtained may further be compressed into tablets or filled in capsules using techniques known in the art. Alternatively, tablets can be prepared by a direct compression technique, using powder blends.

Modified-release pharmaceutical formulations of the present invention may be of the matrix-type or the reservoir-type delivery systems (e.g. osmotic pumps, and the like), which can be further coated to achieve a desired in vitro dissolution profile as desired. Such matrix-type or reservoir-type pharmaceutical formulations comprise niacin, at least one release controlling polymer, and pharmaceutically acceptable excipients. The release controlling polymers can be, for example, in the form of a matrix or a coating. Formulations comprising niacin in modified-release form may comprise, for example, particles of the active agent combined with a release-controlling polymer. The release controlling polymer is a material that permits release of the active agent at a sustained rate in an aqueous medium. The release controlling polymers can be chosen so as to achieve, in combination with the other stated properties, a desired in vitro release rate.

A release-controlling polymer, in the context of the present invention, includes hydrophilic polymers, hydrophobic polymers, delayed release (enteric) polymers, bioadhesive (or mucoadhesive) polymers, hydrophobic substances like waxes and fats, and combinations thereof. The content of release-controlling polymer in the formulations of the present invention may vary from about 1% to about 90%, or from about 5% to about 80%, of the total weight of the formulation.

Useful hydrophilic polymers of various grades include, but are not limited to: cellulose derivatives such as carboxymethyl cellulose, hydroxypropyl methylcellulose (HPMC), hydroxyethyl cellulose, hydroxypropyl cellulose (HPC), cross-linked sodium carboxymethyl cellulose, and cross-linked hydroxypropyl cellulose; carboxymethylamide; potassium methacrylate/divinylbenzene copolymers; polyhydroxyalkyl methacrylates; polyvinylpyrrolidones and cross-linked polyvinylpyrrolidones; high molecular weight polyvinylalcohols; gums such as natural gum, guar, agar, agarose, sodium alginate, carrageenan, fucoidan, furcellaran, laminaran, hypnea, eucheums, gum arabic, gum ghatti, gum karaya, gum tragacanth and locust bean gum; hydrophilic colloids such as alginates; carbomers and polyacrylamides; other substances such as arbinoglactan, pectin, amylopectin, gelatin, N-vinyl lactams, polysaccharides; and the like. Combinations of any two or more of these polymers, and other polymers having the required properties are within the scope of the invention.

Useful hydrophobic polymers or combinations thereof used in various ratios include, but are not limited to: celluloses such as methyl cellulose, ethyl cellulose, cellulose acetates and their derivatives, cellulose acetate phthalate, hydroxypropyl methyl cellulose phthalate, cellulose acylate, cellulose diacylate, cellulose triacylate, cellulose acetate, cellulose diacetate, cellulose triacetate, mono-, di- and tri-cellulose alkanylates, mono-, di-, and tri-cellulose arylates, and mono-, di- and tri-cellulose alkenylates; crosslinked vinylpyrrolidone polymers (crospovidone); polymethacrylic acid based polymers and copolymers such as are sold by Evonik Industries Ltd., Essen, Germany as EUDRAGIT™ (including Eudragit RL and RS, and NE-30D); zein; and aliphatic polyesters. Other classes of polymers, copolymers of these polymers or their mixtures in various ratios and proportions are within the scope of this invention without limitation.

An enteric coating is a coating that prevents release of an active agent until the dosage form reaches a pH environment higher than that of the stomach. A delayed release dosage form comprises active agent and is coated with an enteric polymer. The enteric polymer should be non-toxic and is predominantly soluble in intestinal fluids, but substantially insoluble in the gastric juices. Examples of such delayed release (enteric) polymers include polyvinylacetate phthalate (PVAP), hydroxypropyl methylcellulose acetate succinate (HPMCAS), cellulose acetate phthalate (CAP), methacrylic acid copolymers, hydroxypropyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like), and combinations comprising one or more of the foregoing enteric polymers. Other examples include natural resins, such as shellac, copal collophorium, and combinations comprising one or more of the foregoing polymers. Yet other examples of enteric polymers include synthetic resins bearing carboxyl groups. The methacrylic acid:acrylic acid ethyl ester 1:1 copolymer solid substance of the acrylic dispersion sold under the trade designation “EUDRAGIT L-100-55” is suitable.

A barrier coating may be optionally applied to a core formulation to prevent drug-drug interactions or a drug-enteric coating interactions. It can also impart moisture protection to the core formulation. Non limiting examples of barrier coating materials include hydroxypropyl methylcelluloses (hypromellose or HPMC), hydroxypropylcelluloses and other cellulose derivatives, polyvinyl acetates, polyvinyl alcohols, sugars, amino acids, zein, polyvinylpyrrolidones, guar gum etc. Other inert materials which can act as a barrier to prevent any interaction between the niacin and the enteric polymer or meloxicam, and also between the enteric polymer and meloxicam, are within the scope of the invention without limitation. The determination of the thickness of the barrier coating as well as the viscosity grade of a polymeric material, if used, are within the understanding of a person skilled in the art. Thus, when a polymeric material such as a HPMC is used, a suitable grade could include a low viscosity grade capable of acting as a barrier between the niacin and the enteric coating material without impacting the dissolution and release of the niacin upon contact with an aqueous medium. When a sugar is used as a barrier coating, the thickness of the coating will determine the degree of protection that such a coat will provide as also will the type of sugar. Such and other aspects of selection of a barrier coating are within the scope of understanding of a person skilled in the art of preparation of solid oral dosage forms.

A bioadhesive polymer may be included in oral dosage forms to increase the contact time between the dosage form and the mucosa of a drug-absorbing section of the gastrointestinal tract. Non-limiting examples of bioadhesives include carbomers (various grades), sodium carboxymethyl celluloses, methyl celluloses, polycarbophils (e.g., NOVEON™), hydroxypropyl methylcelluloses, hydroxypropyl celluloses, sodium alginate, sodium hyaluronate, and combinations comprising two or more of the foregoing.

Hydrophobic substances like waxes and fats may have a melting point of about 30° C. to about 200° C., or about 45° C. to about 90° C. Useful hydrophobic substances can include neutral or synthetic waxes, fatty alcohols (such as lauryl, myristyl, stearyl, cetyl or cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising two or more of the foregoing materials. Suitable waxes include beeswax, paraffin wax, carnauba wax, etc., and also synthetic waxes such as for example microcrystalline waxes and other commercially available waxes, castor wax, and wax-like substances, e.g., materials normally solid at room temperature and having a melting point of about 30° C. to about 100° C., and combinations comprising two or more of the foregoing waxes.

Of course, any other release-controlling polymers, which demonstrate similar characteristics, are also acceptable in the working of this invention.

In some embodiments of the present invention, pharmaceutically acceptable excipients serving as pharmaceutically inert cores comprise: insoluble inert materials, such as glass particles/beads or silicon dioxide, calcium phosphate dihydrate, dicalcium phosphate, calcium sulfate dihydrate, microcrystalline cellulose (MCC) or cellulose derivatives; soluble cores such as acid cores like tartaric acid and sugar spheres of sugars like dextrose, lactose, anhydrous lactose, spray-dried lactose, lactose monohydrate, mannitol, starches, sorbitol, sucrose; insoluble inert plastic materials such as spherical or nearly spherical core beads of polyvinyl chloride, polystyrene, or any other pharmaceutically acceptable insoluble synthetic polymeric material; and the like and mixtures thereof.

Modified-release formulations comprising niacin and a release-controlling polymer may be prepared by any suitable technique including those described in detail below. The active agent and a release-controlling polymer may, for example, be prepared by wet granulation techniques, melt extrusion techniques, etc. The active agent in modified-release formulations can include a plurality of substrates comprising the active ingredient, which substrates are coated with a sustained-release coating comprising a release-controlling polymer. The modified-release formulations may thus be made in conjunction with a multiparticulate system, such as beads, ion-exchange resin beads, spheroids, microspheres, seeds, pellets, granules, and other multiparticulate systems in order to obtain a desired modified release of the active agent. The multiparticulate system can be presented in a tablet or capsule or other suitable unit dosage form. In certain cases, more than one multiparticulate system can be used, each exhibiting different characteristics, such as pH dependence of release, time for release in various media (e.g., acidic, basic, simulated intestinal fluids), release in vivo, size, and formulation.

In some cases, a spheronizing agent, together with the active ingredient, can be spheronized to form spheroids. Microcrystalline cellulose and hydrous lactose impalpable are examples of such agents. Additionally (or alternatively), the spheroids can contain a water insoluble polymer, such as an acrylic polymer, an acrylic copolymer, such as a methacrylic acid-ethyl acrylate copolymer, or ethyl cellulose. In this formulation, the release-modifying coating will generally include a water insoluble material such as a wax, either alone or in admixture with a fatty alcohol, or shellac or zein. Spheroids or beads, coated with an active ingredient can be prepared, for example, by dissolving or dispersing the active ingredient in a solvent and then spraying the solution onto a substrate, for example, sugar spheres NF-21, 18/20 mesh, using a Wurster insert. Optionally, additional ingredients are also added prior to coating the beads in order to enhance the active ingredient binding to the substrates, and/or to color the resulting beads, etc. The resulting substrate-active material may optionally be over-coated with a barrier material, to separate the therapeutically active agent from the next coating of material, e.g., a release-controlling polymer.

The pharmaceutical formulations of the present invention can be prepared by various other methods and techniques as known to the skilled person so as to achieve desired in vitro drug release profiles. Specific embodiments of processes comprise any of:

1. Direct compression, using appropriate punches and dies; the punches and dies being fitted to a suitable rotary tableting press.

2. Injection or compression molding using suitable molds fitted to a compression unit.

3. Granulation followed by compression.

4. Extrusion in the form of a paste, into a mold or into an extrudate to be cut into desired lengths.

When particles are made by direct compression, the addition of lubricants may be helpful and sometimes this is important to promote powder flow and to prevent capping of the particle (breaking off of a portion of the particle) when compression pressure is relieved. Typically, lubricants are added in a concentration of from 0.25% to 3% by weight. Additional excipients may be added to enhance powder flowability and reduce adherence.

Oral dosage forms may be prepared to include an effective amount of melt-extruded subunits in the form of multiparticulates within a capsule. For example, a plurality of the melt-extruded muliparticulates can be placed in a gelatin capsule in an amount sufficient to provide an effective release dose when ingested and contacted by gastric fluid. The subunits, e.g., in the form of multiparticulates, can be compressed into oral tablets using conventional tableting equipment using standard techniques.

The formulations may be in the form of microtablets enclosed inside a capsule, e.g., a gelatin capsule. For this, any gelatin capsule employed in the pharmaceutical formulation field can be used, such as the hard gelatin capsules known as CAPSUGEL™, available from Pfizer.

In an embodiment, pharmaceutical formulations of the present invention can be prepared using a granulation process comprising:

a) dissolving or dispersing the active ingredient optionally with binder and/or solubilizer in a solvent;

b) granulating the pharmaceutically acceptable excipient blend with the solution comprising active;

c) drying and lubricating the granules; and

d) compressing the granules into tablets, or alternatively filling into capsules.

In another embodiment, pharmaceutical formulations of the present invention can be prepared using a direct compression process comprising:

a) mixing the active ingredient and a release-controlling polymer, optionally with other pharmaceutically acceptable excipients; and

b) compressing the blend of a) into tablets, or alternatively filling into capsules.

Alternatively, the formulations of the present invention can be prepared by dissolving the active ingredient in a suitable solvent, and layering the dissolved active, optionally with other excipients, onto the surface of inert cores such as tartaric acid and the like as described above. Such drug-layered cores or pellets may further be granulated or coated with a release-controlling polymer to produce pharmaceutical formulations of the present invention.

The granules/beads or tablets or capsules may further be coated with a release-controlling polymer, optionally with other excipients. Such coating can be done using various known techniques such as dip coating, pan coating, fluidized bed coating, and the like.

The residual solvent content of the pharmaceutical formulations, as described herein, may be made low, such as less than about 5000 ppm by weight. The concentration of residual solvents can further be reduced to desired limits, such as are acceptable by regulatory authorities, such as using drying steps.

Surfactants/solubilizers that may be useful in the formulations of the present invention include but are not limited to: anionic surfactants like potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (for example, cholic acid, deoxycholic acid, glycocholic acid, taurocholic acid and glycodeoxycholic acid) and salts thereof (for example, sodium deoxycholate); cationic surfactants like quaternary ammonium compounds (e.g., benzalkonium chloride, cetyltrimethylammonium bromide, lauryldimethylbenzylammonium chloride, acyl carnitine hydrochlorides and alkyl pyridinium halides); nonionic surfactants like polyoxyethylene fatty alcohol ethers (MACROGOL™ and BRIJ™), polyoxyethylene sorbitan fatty acid esters (polysorbates or TWEEN™), polyoxyethylene fatty acid esters (MYRJ™), sorbitan esters (SPAN™), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamer), polaxamines, and the like; and mixtures thereof.

In the context of the present invention, during the processing of the pharmaceutical formulations into finished dosage forms, one or more pharmaceutically acceptable excipients may optionally be used, including but not limited to: diluents such as microcrystalline cellulose (“MCC”), silicified MCC (e.g., PROSOLV™), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; cores/beads such as insoluble inert materials like glass particles/beads or silicon dioxide, calcium phosphate dihydrate, dicalcium phosphate, calcium sulfate dihydrate, microcrystalline cellulose, cellulose derivatives; soluble cores such as sugar spheres of sugars like dextrose, lactose, mannitol, starches, sorbitol, or sucrose; insoluble inert plastic materials such as spherical or nearly spherical core beads of polyvinyl chloride, polystyrene or any other pharmaceutically acceptable insoluble synthetic polymeric material, and the like or mixtures thereof; binders or adherents such as acacia, guar gum, alginic acid, dextrin, maltodextrin, methylcellulose, ethyl cellulose, hydroxyethyl cellulose, hydroxypropyl cellulose (e.g., KLUCEL®), hydroxypropyl methylcellulose (e.g., METHOCEL®), carboxymethyl cellulose sodium, povidone (various grades of KOLLIDON®, PLASDONE®), starch and the like; disintegrants such as carboxymethyl cellulose sodium (e.g., Ac-Di-Sol®, PRIMELLOSE®), crospovidone (e.g., KOLLIDON®, POLYPLASDONE®), povidone K-30, polacrilin potassium, starch, pregelatinized starch, sodium starch glycolate (e.g. EXPLOTAB®), and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like. Solvents that may be used in granulation or layering or coating include water, methanol, ethanol, isopropyl alcohol, acetone, methylene chloride, dichloromethane, and the like and mixtures thereof.

Pharmaceutical formulations of the present invention may further include any one or more of pharmaceutically acceptable glidants, lubricants like sodium stearyl fumarate, opacifiers, colorants, and other commonly used excipients.

The pharmaceutical formulations of the present invention exhibit desired in vivo absorption profiles for the contained actives. The in vivo pharmacokinetic parameters often used to evaluate pharmaceutical formulations upon oral administration include maximum plasma concentration (“Cₘₐₓ”), area under the plasma concentration-time plot curve (“AUC”), plasma concentration at steady state (Cₛₛ), fluctuation index (“FI”), mean residence time (“MRT”), and the like.

The pharmaceutical formulations of the invention may contain one or more active ingredients in addition to niacin and meloxicam. Non-limiting examples of such additional active ingredients include lipid lowering agents, anti-diabetic compounds, other NSAIDs, anti-arrhythmic agents, anti-coagulants, anti-depressants, anti-epileptics, anti-hypertensive agents, anti-neoplastic agents, α-glucosidase inhibitors, erectile dysfunction improvement agents, immunosuppressants, anti-protozoal agents, anti-thyroid agents, anxiolytic agents, sedatives, hypnotics, neuroleptics, beta-blockers, cardiac ionotropic agents, corticosteroids, diuretics, gastrointestinal agents, histamine receptor antagonists, keratolytics, anti-anginal agents, muscle relaxants, nutritional agents, opioid analgesics, protease inhibitors, sex hormones, stimulants, muscle relaxants, anti-osteoporosis agents anti-obesity agents, cognition enhancers, anti-urinary incontinence agents, cholesterol absorption inhibitors, bile acid sequestering agents, and the like. Typically, lipid lowering compounds include statins, fibrates and PPAR agonists. Exemplary statins include atorvastatin, simvastatin, lovastatin, pravastatin, cervastatin, fluvastatin, while fibrates comprise fenofibrate, gemfibrozil, and bezafibrate. Non-limiting examples of DPP IV inhibitors include sitagliptan, vildagliptan, saxagliptan. Typical anti-diabetic compounds include sulfonylureas, meglitinides, DPP-IV inhibitors, biguanides, peroxisome proliferator activated receptor (“PPAR”) agonists, glucose uptake modulators. Cholesterol absorption inhibitors include ezetimibe, and the like. Bile acid sequestering agents include orlistat, and the like. (Praveen: it is a comprehensive list, which includes most of the therapeutic categories, removing the category which is not of interest with respect to present invention may not signify much)

Mention of any specific drug compounds is intended to include any of the alternative forms in which the compounds can be administered, such as their salts, esters, hydrates, solvates, crystalline or amorphous polymorphs, racemic mixtures, enantiomeric isomers, etc.

The pharmaceutical formulations disclosed herein can be advantageously used for the treatment of hyperlipidemia, hypercholesterolemia and mixed dyslipidemia, myocardial infarction, atherosclerotic diseases, for preventing or minimizing niacin-induced flushing, and other such conditions.

The following examples illustrate certain specific aspects and embodiments of the invention and demonstrate the practice and advantages thereof. It is to be understood that the examples are given by way of illustration only and are not intended to limit the scope of the invention in any manner.

### EXAMPLES 1-2

Formulations comprising meloxicam 2.5 mg and modified release niacin 500 mg, in the form of bilayer tablets.

mg/Tablet

| | | |
|---|---|---|
| Ingredient | Example 1 | Example 2 |

LAYER 1
Granulation

| | | |
|---|---|---|
| Niacin | 500 | 500 |
| Polyvinylpyrrolidone (PVP K 30) | 45 | — |
| Water‡ | 100 | — |
| Eudragit L 100 D 55* | — | 45 |
| Isopropyl alcohol‡ | — | 478 |

Base Coating

| | | |
|---|---|---|
| Hypromellose K 100 MCR | 36 | — |
| Water‡ | 1500 | — |

Enteric Coating

| | | |
|---|---|---|
| Eudragit L 100 D 55* | 225 | 261 |
| Isopropyl alcohol‡ | 2250 | 2772 |

LAYER 2

| | | |
|---|---|---|
| Meloxicam | 2.5 | 2.5 |
| Polyvinylpyrrolidone K 30 | 4.25 | 4.25 |
| Water‡ | 50 | 50 |
| Microcrystalline cellulose | 158.25 | 158.25 |
| (Avicel ® PH101)** | | |
| Dibasic calcium phosphate | 44 | 44 |
| Crospovidone | 12.5 | 12.5 |
| Sodium citrate | 20 | 20 |
| Stearic acid | 2.5 | 2.5 |

*Eudragit L 100 D 55 is a co-polymer of methacrylic acid and methacrylates and is a product of Evonik Industries, Germany.
**Avicel ® PH101 is a product of FMC Biopolymer Inc.
‡Evaporates during processing.

Manufacturing Process for Example 1:

1. Polyvinylpyrrolidone was dispersed in water with stirring.

2. Niacin was granulated with dispersion of step 1 using a rapid mixer granulator.

3. The granules of step 2 were dried in fluid bed drier and sifted through a BSS #30 sieve.

4. Hypromellose was dispersed in water with stirring.

5. The granules of step 3 were coated with the dispersion of step 4 using a fluid bed processor.

6. Eudragit was dispersed in isopropyl alcohol with stirring

7. The coated granules of step 5 were coated with Eudragit dispersion of step 6 using a fluid bed processor.

8. Polyvinylpyrrolidone was dispersed in water with stirring.

9. A blend of meloxicam, microcrystalline cellulose, crospovidone and sodium citrate was granulated with the dispersion of step 8 using a rapid mixer granulator.

10. The granules of step 9 were dried in a fluid bed drier and sifted through a BSS #60 sieve.

11. The granules of step 10 were mixed with stearic acid in a blender.

12. The granules of step 7 and step 11 were compressed as a first layer and second layer, respectively, into bilayer tablets using a 22×10 mm punch set on a compression machine to produce an average tablet hardness of 24 to 32 kiloponds (kP).

Manufacturing Process for Example 2:

1. Eudragit was dispersed in isopropyl alcohol with stirring.

2. Niacin particles were granulated using the dispersion of step 1 in a rapid mixer granulator.

3. The granules of step 2 were dried in a fluid bed dryer and sifted through a BSS #30 sieve.

4. Eudragit was dispersed in isopropyl alcohol under stirring.

5. The granules of step 3 were coated with Eudragit dispersion of step 4 using a fluid bed processor.

6. Remaining processing was similar to that described in Example 1, beginning at step 8.

The in vitro release profiles of the meloxicam component of Examples 1-2 tablets were determined using the conditions: phosphate buffer pH 7.5 (900 mL) in USP apparatus 2 (Paddle) from Test 711 “Dissolution” in United States Pharmacopeia 29, United States Pharmacopeial Convention, Inc., Rockville, Md., 2005 (“USP”) with 75 rpm stirring.

Cumulative % of
Meloxicam Dissolved

| Minutes | Example 1 | Example 2 |
|---|---|---|
| 0 | 0 | 0 |
| 10 | 85 | — |
| 30 | 88 | 88 |
| 60 | 95 | 86 |

The in vitro release profiles of the niacin component of Examples 1-2 tablets and a commercial product were determined using the conditions: 0.1 N hydrochloric acid for the initial 2 hours, followed by phosphate buffer pH 6.8 (according to USP Method I for dissolution of enteric coated dosage forms) in USP apparatus 2 (Paddle), from Test 711 “Dissolution” in United States Pharmacopeia 29, United States Pharmacopeial Convention, Inc., Rockville, Md., 2005 (“USP”), with 75 rpm stirring.

| | Cumulative % of Niacin Dissolved | | |
|---|---|---|---|
| Hours | Example 1 | Example 2 | NIASPAN ® 500 mg |
| 0 | 0 | 0 | 0 |
| 0.5 | 12 | 14 | 13 |
| 1 | 20 | 19 | 20 |
| 2 | 28 | 29 | 30 |
| 3 | 37 | 53 | 37 |
| 4 | 55 | 78 | 40 |
| 5 | 72 | 965 | 44 |
| 6 | 84 | 101 | 48 |
| 8 | 99 | 106 | 55 |
| 10 | — | — | 60 |

### EXAMPLE 3

Formulation comprising meloxicam 2.5 mg and modified release niacin 500 mg, in the form of a monolithic tablet.

| | |
|---|---|
| Ingredient | mg/Tablet |

NIACIN GRANULES
Granulation

| | |
|---|---|
| Niacin | 500 |
| Polyvinylpyrrolidone | 45 |
| Water‡ | 100 |

Base Coating

| | |
|---|---|
| Hypromellose K 100 MCR | 36 |
| Water‡ | 1500 |

Enteric Coating

| | |
|---|---|
| Eudragit L 100 D 55 | 225 |
| Isopropyl alcohol‡ | 2900 |

MELOXICAM GRANULES

| | |
|---|---|
| Meloxicam | 2.5 |
| Polyvinylpyrrolidone K 30 | 10 |
| Water‡ | 50 |
| Microcrystalline cellulose | 80 |
| (Avicel ® PH101) | |

EXTRAGRANULAR

| | |
|---|---|
| Stearic acid | 7.5 |

‡Evaporates during processing.

Manufacturing process was similar to that described in Example 1, except the niacin granules and meloxicam granules were mixed and blended with stearic acid in a double cone blender, and further compressed into tablets using a 19×8 mm capsule shape punch set on a compression machine to achieve a hardness of 20-28 kP.

The in vitro release profiles of the niacin component of Examples 3 tablets and a commercial product were determined using the conditions: 0.1 N hydrochloric acid for the initial 2 hours, followed by phosphate buffer pH 6.8 (according to USP Method I for dissolution of enteric coated dosage forms) in USP apparatus 2 (Paddle), from Test 711 “Dissolution” in United States Pharmacopeia 29, United States Pharmacopeial Convention, Inc., Rockville, Md., 2005 (“USP”), with 75 rpm stirring.

| Hours | Cumulative % of Niacin Dissolved |
|---|---|
| 0 | 0 |
| 0.5 | 16 |
| 1 | 24 |
| 2 | 36 |
| 3 | 48 |
| 4 | 63 |
| 5 | 74 |
| 6 | 80 |
| 8 | 97 |
| 10 | 103 |

### EXAMPLES 4-5

Formulations comprising meloxicam 2.5 mg and modified release niacin 500 mg, in the form of a bilayer tablet.

mg/Tablet

| | | |
|---|---|---|
| Ingredient | Example 4 | Example 5 |

LAYER 1
Granulation

| | | |
|---|---|---|
| Niacin | 500 | 500 |
| Glyceryl behenate | — | 50 |
| Hydroxypropyl cellulose LF | 45 | — |
| Water‡ | 100 | — |
| Eudragit L 100 D 55 | — | 45 |
| Isopropyl alcohol‡ | — | 478 |

Base Coating

| | | |
|---|---|---|
| Hydroxypropyl cellulose LF | 36 | — |
| Water‡ | 1500 | — |

Enteric Coating

| | | |
|---|---|---|
| Eudragit L 100 D 55 | 225 | 211 |
| Isopropyl alcohol‡ | 2900 | 2772 |

LAYER 2

| | | |
|---|---|---|
| Meloxicam | 2.5 | 2.5 |
| Pregelatinized starch | 4.25 | 4.25 |
| Water‡ | 50 | 50 |
| Microcrystalline cellulose | 158.25 | 158.25 |
| (Avicel ® PH101) | | |
| Dibasic calcium phosphate | 44 | 44 |
| Crospovidone | 12.5 | 12.5 |
| Sodium citrate | 20 | 20 |
| Stearic acid | 2.5 | 2.5 |

‡Evaporates during processing.

Manufacturing process was similar to that described in Examples 1 and 2.

### EXAMPLE 6

Formulations comprising niacin 500 mg, release delayed using enteric coating.

| Ingredient | mg/Tablet |
|---|---|
| Niacin | 500 |
| Microcrystalline cellulose | 280 |
| (Avicel ® PH101) | |
| Croscarmellose sodium | 25 |
| Eudragit L100-55 | 50 |
| Triethyl Citrate | 12 |
| Isopropyl alcohol‡ | 86.5 |
| Water‡ | 37.5 |
| Talc | 8.7 |

‡Evaporates during processing.

Manufacturing Process:

1. Niacin, microcrystalline cellulose and croscarmellose sodium were mixed together and passed through a BSS #60 sieve.

2. Step 1 mixture was again blended in a blender to attain uniformity.

3. The blend was directly compressed into tablets using 19×8.0 mm punches.

4. Eudragit was dispersed in an isopropyl alcohol-water mixture with stirring, then triethyl citrate and talc were added.

5. Step 4 coating dispersion was coated onto the tablets of step 3 to produce a weight gain of 8%.

The in vitro release profile of the niacin component of Example 6 tablets was determined using the conditions: 0.1 N hydrochloric acid for the initial 2 hours, followed by phosphate buffer pH 6.8, in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Hours | Cumulative % Niacin Dissolved |
|---|---|
| 0 | 0 |
| 0.5 | 0 |
| 1 | 0 |
| 2 | 0 |
| 3 | 102 |
| 4 | 104 |

### EXAMPLE 7

Formulations comprising extended release niacin 500 mg.

| Ingredient | mg/Tablet |
|---|---|
| Niacin | 500 |
| Microcrystalline cellulose | 50 |
| (Avicel ® PH101) | |
| Anhydrous lactose | 50 |
| Eudragit NM 30 D* | 16 |
| Croscarmellose sodium | 5 |
| Stearic acid | 6.5 |

*Eudragit NM 30 D is a co-polymer of methacrylic acid and methacrylates and is a product of Evonik Industries, Germany.

Manufacturing Process:

1. Niacin, microcrystalline cellulose and anhydrous lactose were mixed together and passed through a BSS #60 sieve.

2. Step 1 mixture was again blended in a blender to attain uniformity.

3. The blend of step 2 was granulated in a rapid mixer granulator using Eudragit NM 30 D dispersion.

4. The wet granules of step 3 were dried and were passed through a BSS #24 sieve.

5. Croscarmellose sodium was passed through a BSS #60 sieve and mixed with step 4 granules.

6. Stearic acid was passed through a BSS #60 sieve and mixed with step 5 granules.

7. The blended granules of step 6 were compressed into tablets using 19×8 mm punches.

The in vitro release profile of the niacin component of Example 7 tablets was determined using the conditions: 0.001 N hydrochloric acid (900 mL), pH 3, in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Niacin Dissolved |
|---|---|
| 0 | 0 |
| 10 | 21 |
| 20 | 41 |
| 30 | 59 |
| 60 | 88 |
| 120 | 103 |

### EXAMPLE 8

Formulations comprising extended release niacin 500 mg, release delayed using enteric coating.

| Ingredient | mg/Tablet |
|---|---|
| Niacin | 500 |
| Microcrystalline cellulose | 50 |
| (Avicel ® PH101) | |
| Anhydrous lactose | 50 |
| Eudragit NM 30 D | 16 |
| Croscarmellose sodium | 6.5 |
| Stearic acid | 5 |
| Eudragit L 100-55 | 50 |
| Isopropyl alcohol‡ | 86 |
| HPMC 6 cps | 12 |
| Isopropyl alcohol‡ | 25 |
| Water‡ | 10 |
| Meloxicam | 2.5 |
| HPMC 6 cps | 50 |
| Isopropyl alcohol‡ | 70 |
| Water‡ | 30 |
| HPMC 6 cps | 15 |
| Isopropyl alcohol‡ | 32 |
| Water‡ | 10 |

‡Evaporates during processing.

Manufacturing Process:

1. Niacin, microcrystalline cellulose (Avicel® PH 101) and anhydrous lactose were mixed together and passed through a BSS #60 sieve.

2. Step 1 mixture was again blended in a blender to attain uniformity.

3. The blend of step 2 was granulated in a rapid mixer granulator using Eudragit NM 30 D dispersion.

4. The wet granules of step 3 were dried and were passed through a BSS #24 sieve.

5. Croscarmellose sodium was passed through a BSS #60 sieve and mixed with step 4 granules.

6. Stearic acid was passed through a BSS #60 sieve and mixed with step 5 granules.

7. The blended granules of step 6 were compressed into tablets using 19×8 mm punches.

8. Eudragit L 100-55 solution in isopropyl alcohol was prepared with stirring.

9. The tablets of step 7 were coated with Eudragit L 100-55 solution of step 8, to produce an 8% weight gain.

10. HPMC 6 cps (first quantity) was dissolved in an isopropyl alcohol-water mixture and coated onto the enteric coated niacin tablets to produce a 2% weight gain.

11. Meloxicam and HPMC 6 cps (second quantity) were dissolved in an isopropyl alcohol-water mixture and coated onto step 10 tablets.

12. HPMC 6 cps (third quantity) was dissolved in an isopropyl alcohol-water mixture and coated onto the step 11 tablets to produce a 2% weight gain.

The in vitro release profile of the niacin component of Example 8 tablets was determined using the conditions: 0.1 N hydrochloric acid for the initial 2 hours, followed by phosphate buffer pH 6.8 (according to USP Method I for dissolution of enteric coated dosage forms), in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Niacin Dissolved |
|---|---|
| 0 | 0 |
| 10 | 0.2 |
| 20 | 0.2 |
| 30 | 0.3 |
| 60 | 0.7 |
| 120 | 2 |
| 180 | 97 |
| 240 | 109 |

### EXAMPLE 9

Formulation comprising modified release niacin 500 mg.

| Ingredient | mg/Tablet |
|---|---|
| Niacin | 500 |
| Microcrystalline cellulose | 50 |
| (Avicel ® PH101) | |
| Anhydrous lactose | 25 |
| Eudragit NM 30 D | 16 |
| Stearic acid | 6 |

Manufacturing Process:

1. Niacin, microcrystalline cellulose (Avicel® PH101) and anhydrous lactose were mixed together and passed through a BSS #60 sieve.

2. Step 1 mixture was again blended in a blender to attain uniformity.

3. The blend of step 2 was granulated in a rapid mixer granulator using Eudragit NM 30 D dispersion.

4. The wet granules of step 3 were dried and were passed through a BSS #24 sieve.

5. Stearic acid was passed through a BSS #60 sieve and mixed with step 4 granules.

6. The blended granules of step 5 were compressed into tablets using 19×8 mm punches.

The in vitro release profile of the niacin component of Example 9 tablets was determined using the conditions: 0.001 N HCl (900 mL), in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Niacin Dissolved |
|---|---|
| 0 | 0 |
| 10 | 14 |
| 20 | 19 |
| 30 | 24 |
| 60 | 36 |
| 120 | 56 |
| 180 | 73 |
| 240 | 79 |
| 300 | 91 |
| 360 | 98 |

### EXAMPLE 10

Formulations comprising extended release niacin 500 mg, release delayed using enteric coating.

| Ingredient | mg/Tablet |
|---|---|
| Niacin | 500 |
| Microcrystalline cellulose | 50 |
| (Avicel ® PH112) | |
| Tabletose 70 | 50 |
| Eudragit NM 30 D | 16 |
| Croscarmellose sodium | 5 |
| Stearic acid | 6.5 |
| HPMC 6 cps | 12.5 |
| Isopropyl alcohol‡ | 25 |
| Water‡ | 10 |
| Eudragit L 100-55 | 51 |
| Isopropyl alcohol‡ | 860 |

‡Evaporates during processing.

Manufacturing Process:

1. Niacin, microcrystalline cellulose and Tabletose 70 were mixed together and passed through a BSS #60 sieve.

2. Step 1 mixture was again blended in a blender to attain uniformity.

3. The blend of step 2 was granulated in a rapid mixer granulator using Eudragit NM 30 D dispersion.

4. The wet granules of step 3 were dried and were passed through a BSS #24 sieve.

5. Croscarmellose sodium was passed through a BSS #60 sieve and mixed with step 4 granules.

6. Stearic acid was passed through a BSS #60 sieve and mixed with step 5 granules.

7. The blended granules of step 6 were compressed into tablets using 19×8 mm punches.

8. The tablets of step 7 were coated with a solution of HPMC 6 cps in an isopropyl alcohol-water mixture to produce a 2% weight gain.

9. The tablets of step 8 were coated with a Eudragit L 100-55 solution in isopropyl alcohol to produce an 8% weight gain.

The in vitro release profile of the niacin component of Example 10 tablets was determined using the conditions: 0.1 N hydrochloric acid for the initial 2 hours, followed by phosphate buffer pH 6.8 (according to USP method I for dissolution of enteric coated dosage forms), in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Niacin Dissolved |
|---|---|
| 0 | 0 |
| 10 | 0 |
| 20 | 0 |
| 30 | 0 |
| 60 | 0 |
| 120 | 0 |
| 180 | 97 |
| 240 | 112 |

### EXAMPLE 11

Formulations comprising 2 mg meloxicam and extended release niacin 500 mg, niacin release delayed using enteric coating.

| Ingredient | mg/Tablet |
|---|---|
| Niacin | 500 |
| Microcrystalline cellulose | 50 |
| (Avicel ® PH112) | |
| Tabletose ™ 70 | 50 |
| Eudragit NM 30 D | 16 |
| Croscarmellose sodium | 5 |
| Stearic acid | 6.5 |
| HPMC 6 cps | 18.81 |
| Isopropyl alcohol‡ | 40 |
| Water‡ | 16 |
| HPMC phthalate | 90.41 |
| Triethyl citrate | 10.8 |
| Isopropyl alcohol‡ | 72 |
| Water‡ | 20 |
| HPMC 6 cps | 22.48 |
| Isopropyl alcohol‡ | 47 |
| Water‡ | 20 |
| Meloxicam | 2 |
| HPMC 6 cps | 50 |
| Isopropyl alcohol‡ | 107 |
| Water‡ | 45 |

‡Evaporates during processing.

Manufacturing Process:

1. Niacin, microcrystalline cellulose and Tabletose 70 were mixed together and passed through BSS #60 sieves.

2. Step 1 mixture was again blended in a blender to attain uniformity.

3. The blend of step 2 was granulated in a rapid mixer granulator using Eudragit NM 30 D dispersion.

4. The wet granules of the step 3 were dried and were passed through a BSS #24 sieve.

5. Croscarmellose sodium was passed through a BSS #60 sieve and mixed with step 4 granules.

6. Stearic acid was passed through a BSS #60 sieve and mixed with step 5 granules.

7. The blended granules of step 6 were compressed into tablets using 19×8.5 mm punches.

8. The tablets of 7 were coated with HPMC 6 cps (first quantity) solution prepared in an isopropyl alcohol-water mixture and coated onto the core tablets to produce a 3% weight gain.

9. HPMC phthalate solution was prepared in an isopropyl alcohol-water mixture and triethyl citrate was added.

10. The tablets of step 8 were coated with HPMC phthalate solution of step 9 to produce a 14% weight gain.

11. The tablets of step 10 were further coated with HPMC 6 cps (second quantity) solution prepared in an isopropyl alcohol-water mixture and coated on the tablets to produce a 3% weight gain.

12. Meloxicam and HPMC 6 cps (third quantity) were dissolved in an isopropyl alcohol-water mixture with stirring.

13. The tablets of step 11 were further coated with meloxicam coating solution of step 12 to provide 2 mg of meloxicam per tablet.

The in vitro release profile of the niacin component of Example 11 tablets was determined using the conditions: 0.1 N HCl for 2 hours followed by pH 6.8 buffer (according to USP method I for dissolution of enteric coated dosage forms), in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Niacin Dissolved |
|---|---|
| 0 | 0 |
| 10 | 0 |
| 20 | 0 |
| 30 | 0 |
| 60 | 0 |
| 120 | 0 |
| 180 | 98 |
| 240 | 107 |

The in vitro release profile of the meloxicam component of Example 11 tablets was determined using the conditions: phosphate buffer pH 7.5 (900 mL), in USP apparatus 2 (Paddle) from Test 711 “Dissolution” in United States Pharmacopeia 29, United States Pharmacopeial Convention, Inc., Rockville, Md., 2005 (“USP”), with 75 rpm stirring.

| Minutes | Cumulative % of Meloxicam Dissolved |
|---|---|
| 0 | 0 |
| 10 | 62 |
| 20 | 96 |
| 30 | 106 |

Pharmacokinetic parameters under fasting conditions were determined by administering a single dose of the tablets to four beagle dogs. The pharmacokinetic parameters were calculated after analyzing samples of plasma from the dogs, withdrawn at regular intervals after dosing, for drug content and the mean results are in the tables below.

| Niacin Parameter | Result |
|---|---|
| AUC₀₋ₜ (ng · hour/ml) | 74983 |
| Cₘₐₓ (ng/ml) | 39525 |
| Tₘₐₓ (hours) | 2.00 |

| Meloxicam Parameter | Result |
|---|---|
| AUC₀₋ₜ (ng · hour/ml) | 12943 |
| Cₘₐₓ (ng/ml) | 597 |
| Tₘₐₓ (hours) | 1.63 |

1. A pharmaceutical formulation comprising a therapeutically effective dyslipidemia-treating amount of niacin, and an oxicam NSAID in an amount sufficient to reduce flushing side effects induced by administering the niacin.

2. A pharmaceutical formulation according to claim 1, wherein an oxicam NSAID comprises meloxicam.

3. A pharmaceutical formulation according to claim 2, wherein an amount of meloxicam is less than about 7.5 mg.

4. A pharmaceutical formulation according to claim 3, wherein an amount of niacin is not more than about 2000 mg,

5. A pharmaceutical formulation according to claim 1, wherein niacin and an oxicam NSAID are present in immediate release form.

6. A pharmaceutical formulation according to claim 1, wherein niacin is present in modified release form and an oxicam NSAID is present in immediate release form.

7. A pharmaceutical formulation according to claim 1, providing a substantially simultaneous commencement of release of meloxicam and niacin, following administration.

8. A pharmaceutical formulation according to claim 1, providing a release of meloxicam prior to release of niacin, following administration.

9. A pharmaceutical formulation according to claim 1, releasing no more than about 10 percent of contained niacin, within about 30 minutes after immersion into an aqueous medium having pH values less than about 4.

10. A pharmaceutical formulation according to claim 1, releasing no more than about 20 percent of contained niacin, within about 30 minutes after immersion into an aqueous medium having pH values less than about 4.

11. A pharmaceutical formulation according to claim 1, releasing no more than about 30 percent of contained niacin, within about 30 minutes after immersion into an aqueous medium having pH values less than about 4.

12. A pharmaceutical formulation according to claim 1, releasing at least about 15 percent of contained niacin within about 2 hours following immersion into an aqueous medium having pH values at least about 5.

13. A pharmaceutical formulation according to claim 1, releasing at least about 30 percent of contained meloxicam, within about 30 minutes after immersion into an aqueous medium having pH values at least about 5.

14. A pharmaceutical formulation according to claim 1, releasing at least about 50 percent of contained meloxicam, within about 30 minutes after immersion into an aqueous medium having pH values at least about 5.

15. A pharmaceutical formulation according to claim 1, releasing at least about 70 percent of contained meloxicam, within about 30 minutes after immersion into an aqueous medium having pH values more than about 5.

16. A pharmaceutical formulation comprising: (a) a core comprising niacin; (b) an enteric coating covering the core; and (c) a layer comprising an oxicam NSAID over the enteric coating.

17. A pharmaceutical formulation according to claim 16, further comprising a barrier layer coating between (a) and (b).

18. A pharmaceutical formulation according to claim 16, further comprising a barrier layer coating between (b) and (c).

19. A pharmaceutical formulation according to claim 16, further comprising a barrier layer coating over the layer of (c).

20. A pharmaceutical formulation according to claim 16, wherein a core provides an immediate release or modified release of niacin.

21. A pharmaceutical formulation according to claim 16, providing an immediate release or modified release of an oxicam NSAID.

22. A pharmaceutical formulation according to claim 16, wherein an oxicam NSAID comprises meloxicam.

23. A pharmaceutical formulation containing niacin and an oxicam NSAID, providing a release of at least about 20% of contained oxicam NSAID and no more than about 10% release of contained niacin, within about 30 minutes after immersion into an aqueous medium having pH values less than about 4.

24. A pharmaceutical formulation according to claim 23, providing a release of at least about 20% of contained oxicam NSAID and no more than about 10% release of contained niacin, within about 60 minutes after immersion into an aqueous medium having pH values less than about 4.

25. A pharmaceutical formulation according to claim 23, providing a release of at least about 10% of contained oxicam NSAID and no more than about 5% of contained niacin, within about 30 minutes following immersion into an aqueous medium having pH values less than about 4.

26. A pharmaceutical formulation according to claim 23, providing a release of at least about 30% of contained oxicam NSAID and no more than about 30% of contained niacin, within about 30 minutes following immersion into an aqueous medium having pH values less than about 4.

27. A pharmaceutical formulation according to claim 23, wherein an oxicam NSAID comprises meloxicam.

28. A pharmaceutical formulation according to claim 23, wherein at least about 15% of contained niacin is released within about 2 hours after immersion into an aqueous medium having pH values at least about 5.
